(19) United States
(12) Patent Application Publication
(10) Pub. No.: US 2026/0335379 A1
(43) Pub. Date: Oct. 29, 2026

(54) COMPOSITIONS AND METHODS FOR TREATING MENTAL AND METABOLIC DISORDERS

(71) Applicant: The Regents of the University of California, Oakland, CA (US)

(72) Inventors: Arpana Church, Los Angeles, CA (US); Tien S. Dong, Los Angeles, CA (US)

(21) Appl. No.: 19/486,097

(22) PCT Filed: May 30, 2024
(86) PCT No.: PCT/US2024/031595

§ 371 Date: Nov. 20, 2025

Related U.S. Application Data
(60) Provisional application No. 63/505,308, filed on May 31, 2023.

(51) Int. Cl. A61K 35/741; A61K 31/405

(52) U.S. Cl. CPC A61K 35/741; A61K 31/405

(57) ABSTRACT

The present disclosure relates to bacterial compositions and methods of treating disease and disorders with said bacterial compositions.

### RELATED APPLICATIONS

This application claims the benefit of U.S. Provisional Application No. 63/505,308, filed May 31, 2023, which is hereby incorporated by reference in its entirety.

### GOVERNMENT SUPPORT

This invention was made with government support under DK121025, DK007180, DK106528, and MD015904 awarded by the National Institutes of Health. The government has certain rights in the invention.

### BACKGROUND

Obesity has reached global epidemic proportions and has become one of the leading preventable causes of death in the United States, for example in 2008, an estimated 500 million adults worldwide were considered obese. Obesity is associated with many comorbidities, such as diabetes, cancer(s), premature mortality from cardiovascular disease (CVD), and musculoskeletal disorders.

In addition to the health detriments, the economic and social consequences of obesity are compounding. For instance, in 2011, medical costs associated with treatment of preventable diseases associated with obesity were estimated to increase by $48-66 billion per year in the U.S. alone, with an estimated 65 million more adults to become obese by 2030. Accordingly, there is an ongoing need for new treatments for obesity.

### SUMMARY OF THE INVENTION

Provided herein are methods of treating obesity, food addiction, and/or metabolic disease using compositions and food supplements. In one aspect, the present disclosure provides methods of treating a disease or disorder in a subject comprising conjointly administering to the subject a first bacterial strain from the species of Akkermansia muciniphila and a second bacterial strain from the species of Bacteroides thetaiotaomicron, wherein the disease or disorder is a metabolic disease, obesity, food addiction, a mental health disorder, or a neurodegenerative disease.

In another aspect, the present disclosure provides pharmaceutical compositions comprising a first bacterial strain from the species of Akkermansia muciniphila, a second bacterial strain from the species of Bacteroides thetaiotaomicron, and at least one pharmaceutically acceptable excipient.

In another aspect, the present disclosure provides nutritional supplements comprising a first bacterial strain from the species of Akkermansia muciniphila and a second bacterial strain from the species of Bacteroides thetaiotaomicron.

### BRIEF DESCRIPTION OF THE DRAWINGS

FIG. 1 shows a procedure for administering Akkermansia muciniphila and Bacteroides thetaiotaomicron in a clinical trial to subjects suffering from food addiction and obesity.

### DETAILED DESCRIPTION OF THE INVENTION

Food addiction (FA) is a primary driver of obesity. Overeating and sedentary lifestyles result in a positive energy imbalance, leading to adipose tissue accumulation. According to the Diagnostic and Statistical Manual of Mental Disorders (DSM-IV), an obsessive relationship with food fulfills behavioral criteria for diagnosis of substance addiction: 1) taking the substance in larger amounts than was intended; 2) inability to control its use; 3) taking the substance for a longer period of time than was intended; and 4) continued use despite adverse consequences. The Yale Food Addiction Scale (YFAS) has been developed and validated as a psychometrically sound measure to operationalize human cases of FA using the DSM-IV. YFAS scores were shown to be correlated with neural activation in brain areas contributing to cravings and reduced inhibitory control. Obesity profoundly impacts responsiveness of the brain reward system, altering the brain regions such as the striatum, prefrontal cortical regions, and amygdala-areas likewise altered in drug addiction.

The gut microbiome is gaining recognition as a significant player in the etiology of many diseases as well as obesity physiology. Research has shown that deviation from a core, lean gut microbiome profile is reflective of obesity. In addition to reduced bacterial diversity, there is alteration in bacterial gene representation and phylum-level modifications. Obesity-associated gut microbiomes also change pathways of food metabolism. Correlations between obesity pathophysiology and the gut microbiome have been observed through metagenomic and biochemical analyses, demonstrating that obese gut microbiomes absorb energy at higher efficiencies than lean gut microbiomes. This superfluous harvesting of energy results in accumulation of body fat. Demonstrated herein is the role of gut microbiome and fecal metabolite in relation to food addiction, as well as to addiction centers of the brain.

In one aspect, the present disclosure provides methods of treating a disease or disorder in a subject comprising conjointly administering to the subject a first bacterial strain from the species of Akkermansia muciniphila and a second bacterial strain from the species of Bacteroides thetaiotaomicron, wherein the disease or disorder is a metabolic disease, obesity, food addiction, and a neurodegenerative disease.

In some embodiments, the first bacterial strain and second bacterial strain are administered orally. In some embodiments, the first bacterial strain and second bacterial strain are administered rectally.

In some embodiments, the disease or disorder is a metabolic disease. In some embodiments, the metabolic disease is diabetes (e.g., type II diabetes). In some embodiments, the disease or disorder is obesity. In some embodiments, the disease or disorder is food addiction. In some embodiments, the disease or disorder is a mental health disorder (e.g., depression). In some embodiments, the disease or disorder is a neurodegenerative disease (e.g., Alzheimer's disease).

In some embodiments, the subject has an increased connectivity between the intraparietal sulcus or brain stem and the putamen. In some embodiments, the subject's increased connectivity between the intraparietal sulcus or brain stem and the putamen is determined by MRI (e.g., by Diffusion Tensor Imaging (DTI)). In some embodiments, the compound, composition, or food supplement is only administered if the subject has an increased connectivity between the intraparietal sulcus or brain stem and the putamen.

In some embodiments, the subject has a lower number of bacteria from a bacterial strain selected from the genus of Bacteroides, as compared to an individual with a healthy weight. In some embodiments, the subject has a lower number of bacteria from a bacterial strain selected from the genus of Megamonas, as compared to an individual with a healthy weight. In some embodiments, the subject has a lower number of bacteria from a bacterial strain selected from the genus of Eubacterium, as compared to an individual with a healthy weight. In some embodiments, the subject has a lower number of bacteria from a bacterial strain selected from the genus of Akkermansia, as compared to an individual with a healthy weight. In some embodiments, the subject has a lower number of bacteria from a bacterial strain selected from the phylum of Firmicutes, as compared to an individual with a healthy weight.

In some embodiments, the subject has reduced levels of indolepropionate (e.g., 3-indolepropinoate), as compared to an individual with a healthy weight.

In some embodiments, the subject has increased connectivity within key reward regions of the brain (e.g., between the intraparietal sulcus and putamen), as compared to an individual with healthy weight. In some embodiments, the subject has lower sensitivity to insulin, as compared to an individual with a healthy weight. In some embodiments, the subject has higher plasma cholesterol, as compared to an individual with a healthy weight.

In some embodiments, the subject has diabetes. In some embodiments, the subject is a woman.

In some embodiments, the method further comprises restricting the caloric intake of the subject. In some embodiments, the method further comprises administering an exercise regime for the subject.

In some embodiments, the method further comprises administering indolepropionate or a pharmaceutically acceptable salt thereof. In some preferred embodiments, the indolepropinoate is

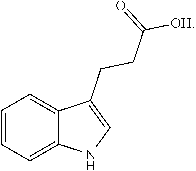 [SMILES: O=C(O)CCc1c[nH]c2ccccc12]

In some embodiments, the indolepropionate is administered orally, rectally, intravenously, intraperitoneally, or subcutaneously. In some preferred embodiments, the indolepropionate is administered orally.

In some embodiments, the indolepropionate and/or bacteria selected from the species of Akkermansia muciniphila and Bacteroides thetaiotaomicron is administered for 30, 40, 45, 50, 60, 70, 80, 90, 100, 110, 120, 130, 140, 150, 160, 170, or 180 days. In some preferred embodiments, the indolepropionate and/or bacteria selected from the species of Akkermansia muciniphila and Bacteroides thetaiotaomicron is administered for 90 days.

In some embodiments, substantially all the bacteria for administration are selected from the species of Akkermansia muciniphila and Bacteroides thetaiotaomicron, or a combination thereof. In some preferred embodiments, the method comprises administering the Akkermansia muciniphila and the Bacteroides thetaiotaomicron in a ratio of about 2:1.

In some embodiments, the method comprises administering about 1×10³ to about 1×10¹³ colony forming units of bacteria selected from species of Akkermansia muciniphila and Bacteroides thetaiotaomicron, or a combination thereof. In some preferred embodiments, the method comprises administering about 1×10⁹ to about 1×10¹¹ colony forming units of bacteria selected from species of Akkermansia muciniphila and Bacteroides thetaiotaomicron, or a combination thereof. In some embodiments, the method comprises administering about 15×10⁹ colony forming units of bacteria selected from species of Akkermansia muciniphila and Bacteroides thetaiotaomicron, or a combination thereof. In some embodiments, the method comprises administering about 10×10⁹ colony forming units of Akkermansia muciniphila and about 5×10⁹ CFU of Bacteroides thetaiotaomicron.

In another aspect, the present disclosure provides pharmaceutical compositions comprising a first bacterial strain from the species of Akkermansia muciniphila, a second bacterial strain from the species of Bacteroides thetaiotaomicron, and at least one pharmaceutically acceptable excipient.

In some embodiments, the pharmaceutical composition comprises live bacteria. In some embodiments, pharmaceutical composition comprises lyophilized bacteria. In some embodiments, the pharmaceutical composition comprises bacterial spores. In some embodiments, the pharmaceutical composition comprises attenuated bacteria.

In some embodiments, the pharmaceutical composition further comprises indolepropionate or a pharmaceutically acceptable salt thereof. In some preferred embodiments, the indolepropinoate is

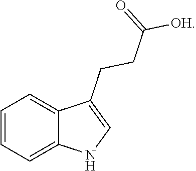 [SMILES: O=C(O)CCc1c[nH]c2ccccc12]

In some embodiments, the pharmaceutical composition is formulated for oral administration. In some embodiments, the pharmaceutical composition is a capsule. In some embodiments, the capsule is enterically coated. In some embodiments, at least 0.1% to at least 99% of the bacteria in the composition are selected from the species of Akkermansia muciniphila and Bacteroides thetaiotaomicron, or a combination thereof.

In some embodiments, substantially all the bacteria in the composition are selected from the species of Akkermansia muciniphila and Bacteroides thetaiotaomicron, or a combination thereof. In some preferred embodiments, the Akkermansia muciniphila and the Bacteroides thetaiotaomicron in a ratio of about 2:1.

In some embodiments, the composition comprises 1×10³ to 1×10¹³ colony forming units of bacteria selected from species of Akkermansia muciniphila and Bacteroides thetaiotaomicron, or a combination thereof. In some preferred embodiments, the composition comprises 1×10⁹ to 1×10¹¹ colony forming units of bacteria selected from species of Akkermansia muciniphila and Bacteroides thetaiotaomicron, or a combination thereof. In some embodiments, the composition comprises about 15×10⁹ CFU of bacteria selected from species of Akkermansia muciniphila and Bacteroides thetaiotaomicron, or a combination thereof. In some preferred embodiments, the composition comprises about 10×10⁹ CFU of Akkermansia muciniphila and about 5×10⁹ CFU of Bacteroides thetaiotaomicron.

In another aspect, the present disclosure provides nutritional supplements comprising a first bacterial strain from the species of Akkermansia muciniphila and a second bacterial strain from the species of Bacteroides thetaiotaomicron.

In some embodiments, the nutritional supplement comprises live bacteria. In some embodiments, the nutritional supplement comprises lyophilized bacteria. In some embodiments, the nutritional supplement comprises bacterial spores. In some embodiments, the nutritional supplement comprises attenuated bacteria.

In some embodiments, the nutritional supplement further comprises indolepropionate or a pharmaceutically acceptable salt thereof. In some preferred embodiments, the indolepropinoate is

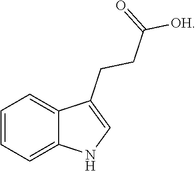 [SMILES: O=C(O)CCc1c[nH]c2ccccc12]

In some embodiments, the nutritional supplement is formulated for oral administration. In some embodiments, the nutritional supplement is a capsule. In some embodiments, the capsule is enterically coated.

In some embodiments, at least 0.1% to at least 99% of the bacteria in the bacterial composition are selected from the species of Akkermansia muciniphila and Bacteroides thetaiotaomicron, or a combination thereof.

In some embodiments, substantially all the bacteria in the bacterial composition are selected from the species of Akkermansia muciniphila and Bacteroides thetaiotaomicron, or a combination thereof. In some embodiments, the Akkermansia muciniphila and the Bacteroides thetaiotaomicron are administered in a ratio of about 2:1.

In some embodiments, the bacterial composition comprises 1×10³ to 1×10¹³ colony forming units of bacteria selected from species of Akkermansia muciniphila and Bacteroides thetaiotaomicron, or a combination thereof. In some embodiments, the bacterial composition comprises 1×10⁹ to 1×10¹¹ colony forming units of bacteria selected from species of Akkermansia muciniphila and Bacteroides thetaiotaomicron, or a combination thereof. In some embodiments, the composition comprises about 15×10⁹ CFU of bacteria selected from species of Akkermansia muciniphila and Bacteroides thetaiotaomicron, or a combination thereof. In some embodiments, the composition comprises about 10×10⁹ CFU of Akkermansia muciniphila and about 5×10⁹ CFU of Bacteroides thetaiotaomicron.

In some embodiments of each of the aspects above, the Akkermansia muciniphila is a strain comprising at least 90%, at least 95%, or at least 99% genomic, 16S, and/or CRISPR sequence identity to the nucleotide sequence of the Akkermansia muciniphila Muc [CIP 107961] (ATCC Deposit Number BAA-835). In some preferred embodiments, the Akkermansia muciniphila is a strain comprising at least 90%, at least 95%, or at least 99% genomic sequence identity to the nucleotide sequence of the Akkermansia muciniphila Muc [CIP 107961] (ATCC Deposit Number BAA-835). In some embodiments, the Akkermansia muciniphila is a strain comprising at least 90%, at least 95%, or at least 99% 16S sequence identity to the nucleotide sequence of the Akkermansia muciniphila Muc [CIP 107961] (ATCC Deposit Number BAA-835).). In some embodiments, the Akkermansia muciniphila is a strain comprising at least 90%, at least 95%, or at least 99% CRISPR sequence identity to the nucleotide sequence of the Akkermansia muciniphila Muc [CIP 107961] (ATCC Deposit Number BAA-835). In some preferred embodiments, the Akkermansia muciniphila strain is Akkermansia muciniphila Muc [CIP 107961] (ATCC Deposit Number BAA-835).

In some embodiments of each of the aspects above, the Bacteroides thetaiotaomicron is a strain comprising at least 90%, at least 95%, or at least 99% genomic, 16S, and/or CRISPR sequence identity to the nucleotide sequence of the Bacteroides thetaiotaomicron VPI 5482 (ATCC Deposit Number 29148). In some preferred embodiments, the Bacteroides thetaiotaomicron is a strain comprising at least 90%, at least 95%, or at least 99% genomic sequence identity to the nucleotide sequence of the Bacteroides thetaiotaomicron VPI 5482 (ATCC Deposit Number 29148). In some embodiments, the Bacteroides thetaiotaomicron is a strain comprising at least 90%, at least 95%, or at least 99% 16S sequence identity to the nucleotide sequence of the Bacteroides thetaiotaomicron VPI 5482 (ATCC Deposit Number 29148). In some embodiments, the Bacteroides thetaiotaomicron is a strain comprising at least 90%, at least 95%, or at least 99% CRISPR sequence identity to the nucleotide sequence of the Bacteroides thetaiotaomicron VPI 5482 (ATCC Deposit Number 29148). In some preferred embodiments, the Bacteroides thetaiotaomicron strain is Bacteroides thetaiotaomicron VPI 5482 (ATCC Deposit Number 29148).

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextran, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable, and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants that may be required.

The ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” it is meant that the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of the compositions and methods of the invention will be that amount of the composition that is the lowest dose effective to produce a therapeutic effect. Such an effective dose will generally depend upon the factors described above.

If desired, the effective daily dose of the composition may be administered as one, two, three, four, five, six or more sub-doses administered separately at appropriate intervals throughout the day, optionally, in unit dosage forms. In certain embodiments of the present invention, the composition may be administered two or three times daily. In preferred embodiments, the composition will be administered once daily.

The patient receiving this treatment is any animal in need, including primates, in particular humans; and other mammals such as equines, cattle, swine, sheep, cats, and dogs; poultry; and pets in general.

In certain embodiments, compositions of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino) ethanol, ethanolamine, ethylenediamine, N-methylglucamine, hydrabamine, 1H-imidazole, lithium, L-lysine, magnesium, 4-(2-hydroxyethyl) morpholine, piperazine, potassium, 1-(2-hydroxyethyl) pyrrolidine, sodium, triethanolamine, tromethamine, and zinc salts. In certain embodiments, contemplated salts of the invention include, but are not limited to, Na, Ca, K, Mg, Zn, or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, l-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvates can be from the solvent of crystallization, inherent in the solvent of preparation or crystallization, or adventitious to such solvent.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Bacterial Compositions

In certain aspects, provided herein are bacterial compositions comprising the species Akkermansia muciniphila and Bacteroides thetaiotaomicron. In some embodiments, the bacterial formulation comprises a bacterium and/or a combination of bacteria described herein and a pharmaceutically acceptable carrier.

In certain embodiments, at least 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% of the bacteria in the bacterial composition are selected from the species of Akkermansia muciniphila and Bacteroides thetaiotaomicron, or a combination thereof. In certain embodiments, substantially all of the bacteria in the bacterial composition are selected from the species of Akkermansia muciniphila and Bacteroides thetaiotaomicron, or a combination thereof. In certain embodiments, the bacterial composition comprises at least 10 colony forming units (CFUs), 100 colony forming units (CFUs), 1×10³ colony forming units (CFUs), 1×10⁴ colony forming units (CFUs), 1×10⁵ colony forming units (CFUs), 5×10⁵ colony forming units (CFUs), 1×10⁶ colony forming units (CFUs), 2×10⁶ colony forming units (CFUs), 3×10⁶ colony forming units (CFUs), 4×10⁶ colony forming units (CFUs), 5×10⁶ colony forming units (CFUs), 6×10⁶ colony forming units (CFUs), 7×10⁶ colony forming units (CFUs), 8×10⁶ colony forming units (CFUs), 9×10⁶ colony forming units (CFUs), 1×10⁷ colony forming units (CFUs), 2×10⁷ colony forming units (CFUs), 3×10⁷ colony forming units (CFUs), 4×10⁷ colony forming units (CFUs), 5×10⁷ colony forming units (CFUs), 6×10⁷ colony forming units (CFUs), 7×10⁷ colony forming units (CFUs), 8×10⁷ colony forming units (CFUs), 9×10⁷ colony forming units (CFUs), 1×10⁸ colony forming units (CFUs), 2×10⁸ colony forming units (CFUs), 3×10⁸ colony forming units (CFUs), 4×10⁸ colony forming units (CFUs), 5×10⁸ colony forming units (CFUs), 6×10⁸ colony forming units (CFUs), 7×10⁸ colony forming units (CFUs), 8×10⁸ colony forming units (CFUs), 9×10⁸ colony forming units (CFUs), 1×10⁹ colony forming units (CFUs), 5×10⁹ colony forming units (CFUs), 1×10¹⁰ colony forming units (CFUs) 5×10¹⁰ colony forming units (CFUs), 1×10¹¹ colony forming units (CFUs) 5×10¹¹ colony forming units (CFUs), 1×10¹² colony forming units (CFUs) 5×10¹² colony forming units (CFUs), 1×10¹³ colony forming units (CFUs) of bacteria selected from species of Akkermansia muciniphila and Bacteroides thetaiotaomicron, or a combination thereof.

In some preferred embodiments, the bacterial composition comprises 1×10⁹ to 1×10¹¹ colony forming units of bacteria selected from species of Akkermansia muciniphila and Bacteroides thetaiotaomicron, or a combination thereof. In some embodiments, the composition comprises about 15×10⁹ CFU of bacteria selected from species of Akkermansia muciniphila and Bacteroides thetaiotaomicron, or a combination thereof. In some embodiments, the composition comprises about 10×10⁹ CFU of Akkermansia muciniphila and about 5×10⁹ CFU of Bacteroides thetaiotaomicron.

The selected dosage level will depend upon a variety of factors including the subject's diet, the route of administration, the time of administration, the residence time of the particular microorganism being employed, the duration of the treatment, other drugs, compounds and/or materials used in combination with the particular composition employed, the age, sex, weight, condition, general health and prior medical history of the patient being treated, and like factors well known in the medical arts.

A physician or veterinarian having ordinary skill in the art can readily determine and prescribe the effective amount of the bacterial composition required. For example, the physician or veterinarian could prescribe and/or administer doses of the bacteria employed in the v composition at levels lower than that required in order to achieve the desired therapeutic effect and gradually increase the dosage until the desired effect is achieved.

In some embodiments, probiotic formulations containing bacteria selected from the species of Akkermansia muciniphila and Bacteroides thetaiotaomicron, or combinations thereof are provided as encapsulated, enteric coated, or powder forms, with doses ranging from 10 to 10¹¹ CFU (e.g., 10¹⁰ CFU). In some embodiments, the composition comprises 10 billion CFU of Akkermansia muciniphila and 5 billion CFU of Bacteroides thetaiotaomicron in food grade “Microbial Freeze Drying Buffer” placed in a glycerin capsule. In some embodiments, the capsule is enteric coated, e.g., for duodenal release at pH 5.5. In some embodiments, the composition comprises a powder of freeze-dried bacteria selected from the species of Akkermansia muciniphila and Bacteroides thetaiotaomicron, or combinations thereof which is deemed to have “Qualified Presumption of Safety” (QPS) status. In some embodiments, the composition is storage-stable at frozen or refrigerated temperature. As used herein, “stably stored” or “storage-stable” refer to a composition in which cells are able to withstand storage for extended periods of time (e.g., at least one month, or two, three, four, six, or twelve months or more) with a less than 95%, 90%, 80%, 70%, 60%, 50%, 40%, 30%, 20%, 15%, 10%, 5%, or 1% decrease in cell viability.

Methods for producing microbial compositions may include three main processing steps. The steps are organism banking, organism production, and preservation. In certain embodiments, a sample that contains an abundance bacteria selected from the species of Akkermansia muciniphila and Bacteroides thetaiotaomicron, or combinations thereof may be cultured by avoiding an isolation step.

For banking, bacteria selected from the species of Akkermansia muciniphila and Bacteroides thetaiotaomicron, or combinations thereof included in the microbial composition may be (1) isolated directly from a specimen or taken from a banked stock, (2) optionally cultured on a nutrient agar or broth that supports growth to generate viable biomass, and (3) the biomass optionally preserved in multiple aliquots in long-term storage.

In embodiments using a culturing step, the agar or broth may contain nutrients that provide essential elements and specific factors that enable growth. An example would be Brain Heart Infusion medium (BHI), a medium composed of 14.5 g/L Casein Peptone, 7.0 g/L Meat Peptone, 6.0 g/L Brain Heart Infusion Solids, 5.0 g/L sodium chloride, 2.5 g/L disodium phosphate, and 2.0 g/L dextrose in demineralized water at pH 7.4A variety of microbiological media and variations are well known in the art. Culture media can be added to the culture at the start, may be added during the culture, or may be intermittently/continuously flowed through the culture. The strains in the bacterial composition may be cultivated alone, as a subset of the microbial composition, or as an entire collection comprising the microbial composition. As an example, a first strain may be cultivated together with a second strain in a mixed continuous culture, at a dilution rate lower than the maximum growth rate of either cell to prevent the culture from washing out of the cultivation.

The inoculated culture is incubated under favorable conditions for a time sufficient to build biomass. For microbial compositions for human use this is often at 37° C. temperature, pH, and other parameters with values similar to the normal human niche. The environment may be actively controlled, passively controlled (e.g., via buffers), or allowed to drift. For example, for anaerobic bacterial compositions, an anoxic/reducing environment may be employed. This can be accomplished by the addition of reducing agents such as cysteine to the broth, and/or stripping it of oxygen. As an example, a culture of a bacterial composition may be grown at 37° C., pH 7, in the medium above, pre-reduced with 1 g/L cysteine-HCl.

When the culture has generated sufficient biomass, it may be preserved for banking. The organisms may be placed into a chemical milieu that protects from freezing (adding ‘cryoprotectants’), drying (‘lyoprotectants’), and/or osmotic shock (‘osmoprotectants’), dispensing into multiple (optionally identical) containers to create a uniform bank, and then treating the culture for preservation. Containers are generally impermeable and have closures that assure isolation from the environment. Cryopreservation treatment is accomplished by freezing a liquid at ultra-low temperatures (e.g., at or below −80° C.). Dried preservation removes water from the culture by evaporation (in the case of spray drying or ‘cool drying’) or by sublimation (e.g., for freeze drying, spray freeze drying). Removal of water improves long-term microbial composition storage stability at temperatures elevated above cryogenic conditions. Microbial composition banking may be done by culturing and preserving the strains individually, or by mixing the strains together to create a combined bank. As an example of cryopreservation, a microbial composition culture may be harvested by centrifugation to pellet the cells from the culture medium, the supernatant decanted and replaced with fresh culture broth containing 15% glycerol. The culture can then be aliquoted into 1 mL cryotubes, sealed, and placed at −80° C. for long-term viability retention. This procedure achieves acceptable viability upon recovery from frozen storage.

Microbial production may be conducted using similar culture steps to banking, including medium composition and culture conditions described above. It may be conducted at larger scales of operation, especially for clinical development or commercial production. At larger scales, there may be several subcultivations of the microbial composition prior to the final cultivation. At the end of cultivation, the culture is harvested to enable further formulation into a dosage form for administration. This can involve concentration, removal of undesirable medium components, and/or introduction into a chemical milieu that preserves the microbial composition and renders it acceptable for administration via the chosen route. The suspension can then be freeze-dried to a powder and titrated.

After drying, the powder may be blended to an appropriate potency, and mixed with other cultures and/or a filler such as microcrystalline cellulose for consistency and ease of handling, and the bacterial composition formulated as provided herein.

In certain aspects, provided are bacterial compositions for administration in subjects. In some embodiments, the bacterial compositions are combined with additional active and/or inactive materials in order to produce a final product, which may be in single dosage unit or in a multi-dose format.

In some embodiments, the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CnH2nOn. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments, the composition comprises at least one lipid. As used herein, a “lipid” includes fats, oils, triglycerides, cholesterol, phospholipids, fatty acids in any form including free fatty acids. Fats, oils, and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments, the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the composition comprises at least one supplemental vitamin. At least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the compositions of the present invention are combined with a carrier (e.g., a pharmaceutically acceptable carrier) which is physiologically compatible with the gastrointestinal tissue of the subject(s) to which it is administered. Carriers can be comprised of solid-based, dry materials for formulation into tablet, capsule, or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. In some embodiments, the carrier is micro-crystalline cellulose (MCC) added in an amount sufficient to complete the one-gram dosage total weight. Carriers can be solid-based dry materials for formulations in tablet, capsule, or powdered form, and can be liquid or gel-based materials for formulations in liquid or gel forms, which forms depend, in part, upon the routes of administration. Typical carriers for dry formulations include, but are not limited to: trehalose, maltodextrin, rice flour, microcrystalline cellulose (MCC) magnesium stearate, inositol, FOS, GOS, dextrose, sucrose, and like carriers. Suitable liquid or gel-based carriers include but are not limited to: water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., approximately pH 7.0). Other carriers or agents for administering the compositions described herein are known in the art.

In some embodiments, the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the bacterial formulation comprises an enteric coating or micro encapsulation. In certain embodiments, the enteric coating or micro encapsulation improves targeting to a desired region of the gastrointestinal tract. For example, in certain embodiments, the bacterial composition comprises an enteric coating and/or microcapsules that dissolve at a pH associated with a particular region of the gastrointestinal tract. In some embodiments, the enteric coating and/or microcapsules dissolve at a pH of about 5.5-6.2 to release in the duodenum, at a pH value of about 7.2-7.5 to release in the ileum, and/or at a pH value of about 5.6-6.2 to release in the colon. Exemplary enteric coatings and microcapsules are described, for example, in U.S. Pat. Pub. No. 2016/0022592, which is hereby incorporated by reference in its entirety.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies. The composition may be a fermented food product, such as, but not limited to, a fermented milk product. Non-limiting examples of fermented food products include kombucha, sauerkraut, pickles, miso, tempeh, natto, kimchi, raw cheese, and yogurt. The composition may also be a food additive, such as, but not limited to, an acidulent (e.g., vinegar). Food additives can be divided into several groups based on their effects. Non-limiting examples of food additives include acidulents (e.g., vinegar, citric acid, tartaric acid, malic acid, fumaric acid, and lactic acid), acidity regulators, anticaking agents, antifoaming agents, foaming agents, antioxidants (e.g., vitamin C), bulking agents (e.g., starch), food coloring, fortifying agents, color retention agents, emulsifiers, flavors and flavor enhancers (e.g., monosodium glutamate), flour treatment agents, glazing agents, humectants, tracer gas, preservatives, stabilizers, sweeteners, and thickeners.

In certain embodiments, the bacteria disclosed herein are administered in conjunction with a prebiotic to the subject. Prebiotics are carbohydrates which are generally indigestible by a host animal and are selectively fermented or metabolized by bacteria. Prebiotics may be short-chain carbohydrates (e.g., oligosaccharides) and/or simple sugars (e.g., mono- and di-saccharides) and/or mucins (heavily glycosylated proteins) that alter the composition or metabolism of a microbiome in the host. The short chain carbohydrates are also referred to as oligosaccharides, and usually contain from 2 or 3 and up to 8, 9, 10, 15 or more sugar moieties. When prebiotics are introduced to a host, the prebiotics affect the bacteria within the host and do not directly affect the host. In certain aspects, a prebiotic composition can selectively stimulate the growth and/or activity of one of a limited number of bacteria in a host. Prebiotics include oligosaccharides such as fructooligosaccharides (FOS) (including inulin), galactooligosaccharides (GOS), trans-galactooligosaccharides, xylooligosaccharides (XOS), chitooligosaccharides (COS), soy oligosaccharides (e.g., stachyose and raffinose) gentiooligosaccharides, isomaltooligosaccharides, mannooligosaccharides, maltooligosaccharides and mannanoligosaccharides. Oligosaccharides are not necessarily single components and can be mixtures containing oligosaccharides with different degrees of oligomerization, sometimes including the parent disaccharide and the monomeric sugars. Various types of oligosaccharides are found as natural components in many common foods, including fruits, vegetables, milk, and honey. Specific examples of oligosaccharides are lactulose, lactosucrose, palatinose, glycosyl sucrose, guar gum, gum Arabic, tagalose, amylose, amylopectin, pectin, xylan, and cyclodextrins. Prebiotics may also be purified or chemically or enzymatically synthesized.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability, and toxicity). In some embodiments, a compound or an agent is administered orally, e.g., to a subject by ingestion. In some embodiments, the orally administered compound or agent is in an extended release or slow-release formulation or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (at world wide web at blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at world wide web at ebi.ac.uk/Tools/psa.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361:1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6:431-440. Konstantinidis K T, Ramette A, and Tiedje J M. 2006. The bacterial species definition in the genomic era. Philos Trans R Soc Lond B Biol Sci 361:1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, the terms “optional” or “optionally” mean that the subsequently described event or circumstance may occur or may not occur, and that the description includes instances where the event or circumstance occurs as well as instances in which it does not.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

“CFU” is a term well-known in the art and refers to a colony forming unit of bacteria.

As used herein the term “healthy weight” refers to an individual with a body mass index between 18.5 and 24.9.

### EXAMPLES

The invention now being generally described, it will be more readily understood by reference to the following examples which are included merely for purposes of illustration of certain aspects and embodiments of the present invention and are not intended to limit the invention.

### Example 1: Preparation and Administration of Blends of Akkermansia muciniphila and Bacteroides thetaiotaomicron for Treating Obesity, Food Addiction, and Metabolic Diseases

There is a strong documented association between the gut microbiome, food addiction, and health. 105 females were examined and evaluated for the connection between the gut microbiome, food addiction, and brain regions. Brain MRI was obtained in all 105 females. Food addiction (FA) was defined using the Yale Food Addiction Scale. Fecal samples were collected for sequencing and metabolomics. Statistical analysis was done using multivariate analyses and machine learning algorithms. The results showed that of the females with obesity, 33.3% exhibited FA as compared to 5.3% and 0.0% of overweight and normal BMI females, respectively (p=0.0001). Based on a multilevel sparse partial least square discriminant analysis, there was a difference in the gut microbiome of females with FA versus those without. Differential abundance testing showed Bacteroides, Megamonas, Eubacterium and Akkermansia were statistically associated with FA (q-value<0.05).

Metabolomics showed that indolepropionic acid was inversely correlated to FA. FA was also correlated with increased connectivity within the brain's reward network, specifically between the intraparietal sulcus, brain stem and putamen. Indolepropionate belongs to a larger class of tryptophan-derived metabolites termed “indoles.” In contrast to other tryptophan derived metabolites (serotonin, kynurenine), which have also been implicated in brain-gut-microbiome interactions in obesity, indoles are the result of exclusively microbial metabolism, in which most undigested dietary tryptophan in the gut is converted to indoles. Indoles have been previously described as being associated with key regions of the extended reward network, as well as being implicated in playing a role in both obesity and FA. Indoles play an important role in modulating kynurenine synthesis, reducing central nervous system inflammation, improving the mucosal intestinal barrier, and altering GLP-1 secretion—all of which have been shown to be disrupted in states of obesity. Although indolepropionate has been less extensively studied, previous work has demonstrated a neuroprotective role of indolepropionate against Alzheimer's disease and neural oxidative stress. Furthermore, a Finnish study of 200 subjects showed that a higher level of serum indolepropionate acid was associated with a reduce risk of type 2 diabetes. These data suggest that indolepropionate may have both local protective effects on intestinal barrier function as well as remote effects on preserving β-cell function and central nervous system inflammation. Additionally, it was found that decreased fecal indolepropionate was associated with not only increased FA behaviors, but that this was related to increased connectivity between key reward regions involving the putamen.

Bacteroides is the major genus belonging to the phylum Bacteroidetes. In both human and mouse studies, a rise in Bacteroidetes is often associated with a leaner phenotype. In studies of bariatric surgery outcomes, subjects that had the most significant weight loss were those that had higher levels of Bacteroides and lower levels of Prevotella. In a prospective study, Bacteroides species were higher in lean individuals and those subjects who were able to achieve weight loss as compared to subjects with obesity. Whether the associations noted between Bacteroides and obesity are causative is still an area of active research. In further studies, Bacteroides was positively associated with indolepropionate and negatively associated with brain regions related to FA. Complete genomic sequencing revealed that Bacteroides thetaiotaomicron was the species most representative of the genus relating to its ability to be protective against obesity. Bacteroides thetaiotaomicron was also seen to affect glutamate pathways.

Akkermansia is another genus that was significantly associated with FA, brain imaging, and fecal metabolites. Akkermansia is a mucin-degrading bacterium that has been extensively studied for its protective role in metabolic syndrome and insulin sensitivity both in human and mouse studies. In a study of 41 females with obesity undergoing calorie restriction, an increase in relative abundance of Akkermansia was associated with improved fasting glucose, waist-to-hip ratio, and subcutaneous adipocyte diameter. This led to a recent phase 1 randomized double-blind, placebo-controlled clinical trial showing that Akkermansia supplementation in obese and overweight volunteers led to improved insulin sensitivity, reduced plasma cholesterol, and a trend towards decreased body weight and fat mass. Analysis of fecal metabolites revealed a negative association between the presence of Akkermansia and levels of indolepropionate and FA. Akkermansia affects GI hormone signaling and adiposity. Akkermansia muciniphila is the type species of Akkermansia, and Akkermansia muciniphila Muc is the type strain.

Microbial analysis showed that Bacteroides and Akkermansia were positively correlated with indolepropionate while bacteria belonging to the phylum Firmicutes were negatively associated. This finding is in line with the numerous studies that have shown an increase in Firmicutes and a decrease in Bacteroides in patients with diabetes, metabolic syndrome, and obesity. In line with the previous fecal microbiome data in FA and indolepropionate, a negative association between Bacteroides and Akkermansia and the connectivity between the putamen and the intraparietal sulcus is seen.

Of note, both Akkermansia muciniphila and Bacteroides thetaiotaomicron are bacteria commonly found in the normal GI tract. Furthermore, several clinical trials have shown the safety of both bacteria as supplements in humans.

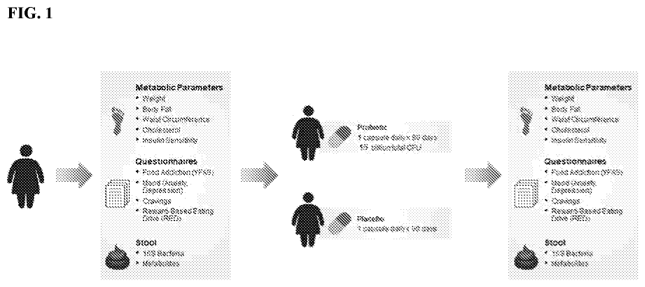

As follows is a protocol for manufacturing a probiotic blend of Akkermansia muciniphila and Bacteroides thetaiotaomicron, and its administration to patients as a treatment for food addiction, obesity, and metabolic disease (FIG. 1). The main aim of the randomized placebo-controlled trial is to determine if a novel blend of a probiotic consisting of Akkermansia muciniphila and Bacteroides thetaiotaomicron can help patients lose more weight. All participants will go on a calorie restricted diet in addition to taking a probiotic or placebo supplement.

### Manufacturing Probiotic Blend

Probiotic capsules comprising Akkermansia muciniphila and Bacteroides thetaiotaomicron can be prepared with known methods. The following procedures are done in sterile conditions to avoid any bacteria contamination.

Cultures of Akkermansia muciniphila and Bacteroides thetaiotaomicron can be inoculated from 1 mL stocks and into 15 mL of Brain Heart Infusion (BHI) sterile growth media. The strains can then be grown in an anaerobic chamber with a gas composition of 90% N₂, 5% H₂, and 5% CO₂ while being shaken at 37° C. Growth can be assessed by measuring optical density readings at 600 nm or using a colony forming unit assay, comprising a serial dilution and growing the bacteria on BHI Agar plates. These assays will be used to determine the concentration of bacterial culture.

Sufficient culture will be prepared to produce 100 capsules at a time. 10 billion CFU of Akkermansia and 5 billion CFU of Bacteroides will be needed per capsule to make the 15 billion CFU capsule. Therefore, based on the CFU/ml calculated above of inoculating 1 ml of stock bacteria with 15 ml BHI sterile broth, calculate the new inoculum. For example, if 1 ml of stock with 15 ml of BHI sterile broth leads to 1×10¹⁰ CFU/ml after 24 hours of growth, then to make 100 capsules of Akkermansia, 10¹² CFU will be needed. This will require 100 ml of culture at 10¹⁰ CFU/ml, so 6.25 ml of stock can be added to 93.75 ml of sterile BHI broth and grown for 24 hours.

Next, the cell culture will be freeze-dried. Under anaerobic and sterile conditions, cell cultures of the appropriate density will be aliquoted into appropriate volumes (such as 50 ml), concentrated via centrifuge at 4° C., resuspended with sterile buffer (such as 15 ml), concentrated via centrifuge at 4° C., resuspended with Microbial Freeze-Drying Buffer (Note: Microbial freeze-drying buffer is made of food grade plant-based protein and sugars in sterile water) to reach the desired cell density, and aliquoted into containers suitable for freeze drying. The resuspended cells will then be frozen at −80° C. for 5 hours, and then placed into the freeze dryer. Cells will be exposed to 0.63 mbar for 12 hours at 0° C., followed by an exposure to 0.63 mbar for 3 hours at 20° C. Each vial will then be sealed under vacuum, and samples will be stored at −80° C. for long term storage or 4° C. for immediate use. °

Packaging the probiotic capsule can be completed as follows. Using the capsule loader, the contents of one freeze dry vial of Akkermansia and one freeze dry vial of Bacteroides will be combined into each of the 100 capsules (glycerin capsules, size 0) using a sterile funnel. Capsules will be stored at 4° C. after preparation.

The quality of each batch can be assessed as follows. Add the end of each batch process, the number of CFU per capsule and the bacterial strain 1 day after the capsule was made will be tested. One capsule will be added to 2 mL of sterile BHI media to reconstitute the bacteria at room temperature. After 4 hours of incubation, CFU counting via serial dilution and plating on BHI Agar will be performed. Agar plates will be allowed to incubate at 37° C. in anaerobic conditions for 24-48 hours before dilutions are counted. The total number of bacteria should be within an order of magnitude of the desired concentration. A second capsule will be reconstituted in BHI media. After 4 hours, the DNA will be extracted using known methods, and real-time PCR using genus-specific primers will be performed, along with a standard curve, to confirm the quantity of bacteria. This process can be completed on capsules stored at 4° C. for 45 days to determine shelf stability of the capsules at 45 days.

### Administering Probiotic Blend to Subjects

The probiotic capsules can be delivered to participants of a clinical trial. The outline of this trial is shown in FIG. 1.

To begin, Participants will be screened using a telephone script. If participants meet the inclusion and exclusion criteria and are willing to participate, the participant will then be scheduled for their baseline visit. The inclusion criteria are as follows: subjects must be between 18 and 50 years of age and of a BMI between 25-40. The exclusion criteria are as follows: subjects must not have co-morbidities (including but not limited to Type I diabetes, have vascular disease, have recent drastic weight loss, engage in frequent strenuous exercise, have undergone abdominal surgeries including weight loss surgery or partial/complete resection of stomach or bowel, have untreated thyroid disease, have neurological disease, have major medical conditions a reasonable physician feels would put the subject at risk or interfere with data collection, have chronic pain, have a diagnosed DSM IV active psychiatric illness including eating disorders (must be active or present for at least 2 years), use medications known to affect hunger/satiety/appetite, be pregnant, be lactating, be less than 6 months post-partum, be planning to get pregnant during the study period, have used oral or intravenous antibiotics within the past 3 months, have used of probiotics within the past month, engage in heavy tobacco use, engage in heavy alcohol use, engage in heavy drug use, have had recent significant changes in diet, have had recent significant weight loss of more than 10 pounds in the last 2 months, and any other criteria up to the physicians' discretion.

At their baseline visit, participants will be again instructed on the study protocol. They will then sign the consent form and complete a W-9 form for payment. The following will be measured: height, weight, waist and hip circumference, BIA. The following questionnaires will be administered: Reward-Based Eating Drive (RED), Food Addiction (YFAS), quality of life (QOL), hospital anxiety and depression scale (HADS), Food Cravings, Dietary food questionnaire. Blood will then be collected via vein puncture and 5 ml will be collected into a lavender top tube. A sampling of participants will receive fMRI to monitor changes in brain activity. Surveys to sample mental health, cognition, and quality of life will also be administered. Participants will then be given an at home stool collection kit with a self-addressed stamped envelope for return.

Participants will then be randomly assigned to the placebo vs 15 billion CFU per capsule groups in a 1:1 fashion using block randomization. All participants will be instructed to undergo a calorie restriction diet. Basal metabolic rate will be calculated using the BIA machine. Basal metabolic rate will be multiplied by 1.1 to account of activity of daily living. 500 will then be subtracted from this number and this will be their goal daily calorie intake. Patients will be instructed on the use of a free phone calorie counter app and given personal advice by a nutritionist or physician. All parties will be blinded to the patient's grouping. Participants will be given a 45-day supply of either the probiotic or placebo. Both groups will be instructed to refrigerate their supplement and instructed to take 1 pill twice a day (i.e., 90 pills per bottle). A follow up visit will then be scheduled in about 1.5 months and the participant will be instructed to return with their pill bottle to measure compliance.

At the interim visit that is scheduled about 1.5 months into the study, participants will return their pill bottle and the number of pills left over will be recorded. Participants will be given another 45-day supply of their respective supplements. Blood will then be collected via vein puncture and 5 ml will be collected into a lavender top tube. A follow up visit will then be scheduled in about 1.5 months for their final visit and the participant will be instructed to return with their pill bottle to measure compliance.

At the final visit scheduled 3 months (90 days) after the start of the study, participants will return their pill bottle and the number of pills left over will be recorded. The following will be measured: height, weight, waist and hip circumference, BIA. The following questionnaires will be administered: RED, YFAS, QOL, HADS, Food Cravings, Dietary food questionnaire. Blood will then be collected via vein puncture and 5 ml will be collected into a lavender top tube. A sampling of participants will receive fMRI to monitor changes in brain activity. Surveys to sample mental health, cognition, and quality of life will also be administered. Participants will then be given an at home stool collection kit with a self-addressed stamped envelope for return.

All data from the participants will then be analyzed to determine the effects of the probiotic supplement routine on metabolic parameters, food addiction, mood, cravings, and reward-based eating drives. Additionally, the stool samples from the beginning and end of the trial will be analyzed for the relative abundance of bacterial species since the start of the trial. Finally, the stool samples will be analyzed for metabolites whose prevalence may have changed over the course of the trial.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of treating a disease or disorder in a subject comprising conjointly administering to the subject a first bacterial strain from the species of Akkermansia muciniphila and a second bacterial strain from the species of Bacteroides thetaiotaomicron, wherein the disease or disorder is a metabolic disease, obesity, food addiction, a mental health disorder, or a neurodegenerative disease.

2. The method of claim 1, wherein the Akkermansia muciniphila is a strain comprising at least 90% genomic, 16S, and/or CRISPR sequence identity to the nucleotide sequence of the Akkermansia muciniphila Muc [CIP 107961] (ATCC Deposit Number BAA-835).

3. The method of claim 2, wherein the Akkermansia muciniphila is a strain comprising at least 95% genomic, 16S, and/or CRISPR sequence identity to the nucleotide sequence of the Akkermansia muciniphila Muc [CIP 107961] (ATCC Deposit Number BAA-835).

4. The method of claim 3, wherein the Akkermansia muciniphila is a strain comprising at least 99% genomic, 16S, and/or CRISPR sequence identity to the nucleotide sequence of the Akkermansia muciniphila Muc [CIP 107961] (ATCC Deposit Number BAA-835).

5. The method of claim 4, wherein the Akkermansia muciniphila is Akkermansia muciniphila Muc [CIP 107961] (ATCC Deposit Number BAA-835).

6. The method of claim 2, wherein the Akkermansia muciniphila is a strain comprising at least 90% genomic sequence identity to the nucleotide sequence of the Akkermansia muciniphila Muc [CIP 107961] (ATCC Deposit Number BAA-835).

7. The method of claim 2, wherein the Akkermansia muciniphila is a strain comprising at least 90% 16S sequence identity to the nucleotide sequence of the Akkermansia muciniphila Muc [CIP 107961] (ATCC Deposit Number BAA-835).

8. The method of claim 2, wherein the Akkermansia muciniphila is a strain comprising at least 90% CRISPR sequence identity to the nucleotide sequence of the Akkermansia muciniphila Muc [CIP 107961] (ATCC Deposit Number BAA-835).

9. The method of claim 3, wherein the Akkermansia muciniphila is a strain comprising at least 95% genomic sequence identity to the nucleotide sequence of the Akkermansia muciniphila Muc [CIP 107961] (ATCC Deposit Number BAA-835).

10. The method of claim 3, wherein the Akkermansia muciniphila is a strain comprising at least 95% 16S sequence identity to the nucleotide sequence of the Akkermansia muciniphila Muc [CIP 107961] (ATCC Deposit Number BAA-835).

11. The method of claim 3, wherein the Akkermansia muciniphila is a strain comprising at least 95% CRISPR sequence identity to the nucleotide sequence of the Akkermansia muciniphila Muc [CIP 107961] (ATCC Deposit Number BAA-835).

12. The method of claim 4, wherein the Akkermansia muciniphila is a strain comprising at least 99% genomic sequence identity to the nucleotide sequence of the Akkermansia muciniphila Muc [CIP 107961] (ATCC Deposit Number BAA-835).

13. The method of claim 4, wherein the Akkermansia muciniphila is a strain comprising at least 99% 16S sequence identity to the nucleotide sequence of the Akkermansia muciniphila Muc [CIP 107961] (ATCC Deposit Number BAA-835).

14. The method of claim 4, wherein the Akkermansia muciniphila is a strain comprising at least 99% CRISPR sequence identity to the nucleotide sequence of the Akkermansia muciniphila Muc [CIP 107961] (ATCC Deposit Number BAA-835).

15. The method of any one of claims 1-14, wherein the Bacteroides thetaiotaomicron is a strain comprising at least 90% genomic, 16S, and/or CRISPR sequence identity to the nucleotide sequence of the Bacteroides thetaiotaomicron VPI 5482 (ATCC Deposit Number 29148).

16. The method of claim 15, wherein the Bacteroides thetaiotaomicron is a strain comprising at least 95% genomic, 16S, and/or CRISPR sequence identity to the nucleotide sequence of the Bacteroides thetaiotaomicron VPI 5482 (ATCC Deposit Number 29148).

17. The method of claim 16, wherein the Bacteroides thetaiotaomicron is a strain comprising at least 99% genomic, 16S, and/or CRISPR sequence identity to the nucleotide sequence of the Bacteroides thetaiotaomicron VPI 5482 (ATCC Deposit Number 29148).

18. The method of claim 17, wherein the Bacteroides thetaiotaomicron is Bacteroides thetaiotaomicron VPI 5482 (ATCC Deposit Number 29148).

19. The method of claim 15, wherein the Bacteroides thetaiotaomicron is a strain comprising at least 90% genomic sequence identity to the nucleotide sequence of the Bacteroides thetaiotaomicron VPI 5482 (ATCC Deposit Number 29148).

20. The method of claim 15, wherein the Bacteroides thetaiotaomicron is a strain comprising at least 90% 16S sequence identity to the nucleotide sequence of the Bacteroides thetaiotaomicron VPI 5482 (ATCC Deposit Number 29148).

21. The method of claim 15, wherein the Bacteroides thetaiotaomicron is a strain comprising at least 90% CRISPR sequence identity to the nucleotide sequence of the Bacteroides thetaiotaomicron VPI 5482 (ATCC Deposit Number 29148).

22. The method of claim 16, wherein the Bacteroides thetaiotaomicron is a strain comprising at least 95% genomic sequence identity to the nucleotide sequence of the Bacteroides thetaiotaomicron VPI 5482 (ATCC Deposit Number 29148).

23. The method of claim 16, wherein the Bacteroides thetaiotaomicron is a strain comprising at least 95% 16S sequence identity to the nucleotide sequence of the Bacteroides thetaiotaomicron VPI 5482 (ATCC Deposit Number 29148).

24. The method of claim 16, wherein the Bacteroides thetaiotaomicron is a strain comprising at least 95% CRISPR sequence identity to the nucleotide sequence of the Bacteroides thetaiotaomicron VPI 5482 (ATCC Deposit Number 29148).

25. The method of claim 17, wherein the Bacteroides thetaiotaomicron is a strain comprising at least 99% genomic sequence identity to the nucleotide sequence of the Bacteroides thetaiotaomicron VPI 5482 (ATCC Deposit Number 29148).

26. The method of claim 17, wherein the Bacteroides thetaiotaomicron is a strain comprising at least 99% 16S sequence identity to the nucleotide sequence of the Bacteroides thetaiotaomicron VPI 5482 (ATCC Deposit Number 29148).

27. The method of claim 17, wherein the Bacteroides thetaiotaomicron is a strain comprising at least 99% CRISPR sequence identity to the nucleotide sequence of the Bacteroides thetaiotaomicron VPI 5482 (ATCC Deposit Number 29148).

28. The method of any one of claims 1-27, wherein the first bacterial strain and second bacterial strain are administered orally.

29. The method of any one of claims 1-27, wherein the first bacterial strain and second bacterial strain are administered rectally.

30. The method of any one of claims 1-29, wherein the disease or disorder is a metabolic disease.

31. The method of claim 30, wherein the metabolic disease is diabetes (e.g., type II diabetes).

32. The method of any one of claims 1-31, wherein the disease or disorder is obesity.

33. The method of any one of claims 1-32, wherein the disease or disorder is food addiction.

34. The method of any one of claims 1-33, wherein the disease or disorder is a mental health disorder (e.g., depression).

35. The method of any one of claims 1-34, wherein the disease or disorder is a neurodegenerative disease (e.g., Alzheimer's disease).

36. The method of any one of claims 1-35, wherein the subject has an increased connectivity between the intraparietal sulcus or brain stem and the putamen.

37. The method of claim 36, wherein the subject's increased connectivity between the intraparietal sulcus or brain stem and the putamen is determined by MRI (e.g., by Diffusion Tensor Imaging (DTI)).

38. The method of any one of claims 1-37, wherein the compound, composition, or food supplement is only administered if the subject has an increased connectivity between the intraparietal sulcus or brain stem and the putamen.

39. The method of any one of claims 1-38, wherein the subject is a woman.

40. The method of any one of claims 1-39, wherein the subject has a lower number of bacteria from a bacterial strain selected from the genus of Bacteroides, as compared to an individual with a healthy weight.

41. The method of any one of claims 1-40, wherein the subject has a lower number of bacteria from a bacterial strain selected from the genus of Megamonas, as compared to an individual with a healthy weight.

42. The method of any one of claims 1-41, wherein the subject has a lower number of bacteria from a bacterial strain selected from the genus of Eubacterium, as compared to an individual with a healthy weight.

43. The method of any one of claims 1-42, wherein the subject has a lower number of bacteria from a bacterial strain selected from the genus of Akkermansia, as compared to an individual with a healthy weight.

44. The method of any one of claims 1-43, wherein the subject has a lower number of bacteria from a bacterial strain selected from the phylum of Firmicutes, as compared to an individual with a healthy weight.

45. The method of any one of claims 1-44, wherein the subject has increased connectivity within key reward regions of the brain (e.g., between the intraparietal sulcus and putamen), as compared to an individual with healthy weight

46. The method of any one of claims 1-45, wherein the subject has reduced levels of indolepropionate (e.g., 3-indolepropinoate), as compared to an individual with a healthy weight.

47. The method of any one of claims 1-46, wherein the subject has lower sensitivity to insulin, as compared to an individual with a healthy weight.

48. The method of any one of claims 1-47, wherein the subject has higher plasma cholesterol, as compared to an individual with a healthy weight.

49. The method of any one of claims 1-48, wherein the subject has diabetes.

50. The method of any one of claims 1-49, wherein the method further comprises restricting the caloric intake of the subject.

51. The method of any one of claims 1-50, wherein the method further comprises administering an exercise regime for the subject.

52. The method of any one of claims 1-51, wherein the method further comprises administering indolepropionate or a pharmaceutically acceptable salt thereof.

53. The method of claim 52, wherein the indolepropinoate is

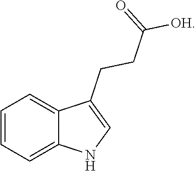 [SMILES: O=C(O)CCc1c[nH]c2ccccc12]

54. The method of claim 52 or 53, wherein the indolepropionate is administered orally, rectally, intravenously, intraperitoneally, or subcutaneously; preferably wherein the indolepropionate is administered orally.

55. The method of any one of claims 1-54, wherein substantially all the bacteria for administration are selected from the species of Akkermansia muciniphila and Bacteroides thetaiotaomicron, or a combination thereof.

56. The method of any one of claims 1-55, the method comprises administering the Akkermansia muciniphila and the Bacteroides thetaiotaomicron in a ratio of about 2:1.

57. The method of any one of claims 1-56, the method comprises administering about 1×10³ to about 1×10¹³ colony forming units of bacteria selected from species of Akkermansia muciniphila and Bacteroides thetaiotaomicron, or a combination thereof.

58. The method of claim 57, the method comprises administering about 1×10⁹ to about 1×10¹¹ colony forming units of bacteria selected from species of Akkermansia muciniphila and Bacteroides thetaiotaomicron, or a combination thereof.

59. The method of claim 58, the method comprises administering about 15×10⁹ colony forming units of bacteria selected from species of Akkermansia muciniphila and Bacteroides thetaiotaomicron, or a combination thereof.

60. The method of claim 56 or 57, the method comprises administering about 10×10⁹ colony forming units of Akkermansia muciniphila and about 5×10⁹ CFU of Bacteroides thetaiotaomicron.

61. A pharmaceutical composition comprising a first bacterial strain from the species of Akkermansia muciniphila, a second bacterial strain from the species of Bacteroides thetaiotaomicron, and at least one pharmaceutically acceptable excipient.

62. The pharmaceutical composition of claim 61, wherein the pharmaceutical composition comprises live bacteria.

63. The pharmaceutical composition of claim 61 or 62, wherein the pharmaceutical composition comprises lyophilized bacteria.

64. The pharmaceutical composition of any one of claims 61-63, wherein the pharmaceutical composition comprises bacterial spores.

65. The pharmaceutical composition of any one of claims 61-64, wherein the pharmaceutical composition comprises attenuated bacteria.

66. The pharmaceutical composition of any one of claims 61-65, further comprising indolepropionate or a pharmaceutically acceptable salt thereof.

67. The pharmaceutical composition of claim 66, wherein the indolepropinoate is

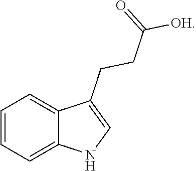 [SMILES: O=C(O)CCc1c[nH]c2ccccc12]

68. The pharmaceutical composition of any one of claims 61-67, wherein the pharmaceutical composition is formulated for oral administration.

69. The pharmaceutical composition of any one of claims 61-68, wherein the pharmaceutical composition is a capsule.

70. The pharmaceutical composition of claim 69, wherein the capsule is enterically coated.

71. The pharmaceutical composition of any one of claims 61-70, wherein at least 0.1% to at least 99% of the bacteria in the composition are selected from the species of Akkermansia muciniphila and Bacteroides thetaiotaomicron, or a combination thereof.

72. The pharmaceutical composition of any one of claims 61-71, wherein substantially all the bacteria in the composition are selected from the species of Akkermansia muciniphila and Bacteroides thetaiotaomicron, or a combination thereof.

73. The pharmaceutical composition of any one of claims 61-72, wherein the Akkermansia muciniphila and the Bacteroides thetaiotaomicron in a ratio of about 2:1.

74. The pharmaceutical composition of any one of claims 61-73, wherein the composition comprises 1×10³ to 1×10¹³ colony forming units of bacteria selected from species of Akkermansia muciniphila and Bacteroides thetaiotaomicron, or a combination thereof.

75. The pharmaceutical composition of any one of claims 61-74, wherein the composition comprises 1×10⁹ to 1×10¹¹ colony forming units of bacteria selected from species of Akkermansia muciniphila and Bacteroides thetaiotaomicron, or a combination thereof.

76. The pharmaceutical composition of any one of claims 61-75, wherein the composition comprises about 15×10⁹ CFU of bacteria selected from species of Akkermansia muciniphila and Bacteroides thetaiotaomicron, or a combination thereof.

77. The pharmaceutical composition of any one of claims 61-76, wherein the composition comprises about 10×10⁹ CFU of Akkermansia muciniphila and about 5×10⁹ CFU of Bacteroides thetaiotaomicron.

78. A nutritional supplement comprising a first bacterial strain from the species of Akkermansia muciniphila and a second bacterial strain from the species of Bacteroides thetaiotaomicron.

79. The nutritional supplement of claim 65, wherein the nutritional supplement comprises live bacteria.

80. The nutritional supplement of claim 65, wherein the nutritional supplement comprises lyophilized bacteria.

81. The nutritional supplement of claim 65, wherein the nutritional supplement comprises bacterial spores.

82. The nutritional supplement of claim 65, wherein the nutritional supplement comprises attenuated bacteria.

83. The nutritional supplement of any one of claims 65-69, further comprising indolepropionate or a pharmaceutically acceptable salt thereof.

84. The nutritional supplement of claim 70, wherein the indolepropinoate is

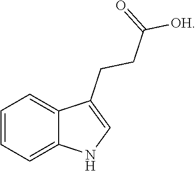 [SMILES: O=C(O)CCc1c[nH]c2ccccc12]

85. The nutritional supplement of any one of claims 78-84, wherein the nutritional supplement is formulated for oral administration.

86. The nutritional supplement of any one of claims 78-85, wherein the nutritional supplement is a capsule.

87. The nutritional supplement of claim 86, wherein the capsule is enterically coated.

88. The nutritional supplement of any one of claims 78-87, wherein at least 0.1% to at least 99% of the bacteria in the bacterial composition are selected from the species of Akkermansia muciniphila and Bacteroides thetaiotaomicron, or a combination thereof.

89. The nutritional supplement of any one of claims 78-88, wherein substantially all the bacteria in the bacterial composition are selected from the species of Akkermansia muciniphila and Bacteroides thetaiotaomicron, or a combination thereof.

90. The nutritional supplement of any one of claims 78-89, wherein the Akkermansia muciniphila and the Bacteroides thetaiotaomicron are administered in a ratio of about 2:1.

91. The nutritional supplement of any one of claims 78-90, wherein the bacterial composition comprises 1×10³ to 1×10¹³ colony forming units of bacteria selected from species of Akkermansia muciniphila and Bacteroides thetaiotaomicron, or a combination thereof.

92. The nutritional supplement of any one of claims 78-91, wherein the bacterial composition comprises 1×10⁹ to 1×10¹¹ colony forming units of bacteria selected from species of Akkermansia muciniphila and Bacteroides thetaiotaomicron, or a combination thereof.

93. The nutritional supplement of any one of claims 78-92, wherein the composition comprises about 15×10⁹ CFU of bacteria selected from species of Akkermansia muciniphila and Bacteroides thetaiotaomicron, or a combination thereof.

94. The nutritional supplement of any one of claims 78-93, wherein the composition comprises about 10×10⁹ CFU of Akkermansia muciniphila and about 5×10⁹ CFU of Bacteroides thetaiotaomicron.
